(19) United States
(12) Patent Application Publication
(10) Pub. No.: US 2005/0215627 A1
(43) Pub. Date: Sep. 29, 2005

(54) Application of substances acting as cascade inhibitors of the Raf/MEK/ERK signaling cascade for the production of a drug against DNA and RNA viruses

(75) Inventors: Stephan Ludwig, Wurzburg (DE); Stephan Pleschka, Giessen (DE)

Correspondence Address: Mark D. Wieczorek, Post Office Box 70072, San Diego, CA, 91267, US

(73) Assignee: MedInnova Gesellschaft fur Medizinische Innovationen aus Akademischer Forschung mbH

(21) Appl. No.: 10/970,118

(22) Filed: Oct. 21, 2004

Related U.S. Application Data
(63) Continuation of application No. 10/240,904, filed on Oct. 4, 2002, now abandoned.

(30) Foreign Application Priority Data
Apr. 7, 2000 (DE) 100 17 480.9

(51) Int. Cl. A61K 31/343; A61K 31/275; A61K 31/165; A61K 31/137

(52) U.S. Cl. 514/469; 514/521; 514/620; 514/649

(57) ABSTRACT

The invention consists in that substances acting as cascade inhibitors of the Raf/MEK/ERK signaling pathway, in particular MEK inhibitors, are used for the production of a drug for the preventive and antiviral therapy against DNA and RNA viruses, in particular against intranuclear-replicating negative strand RNA viruses, for instance influenza or Borna disease viruses.

The present invention is based on the first observation that an infection with the intranuclear-replicating negative strand viruses, in particular influenza A virus and Borna disease virus (BDV), will lead to an activation of the Raf/MEK/ERK cascade, and that surprisingly the inhibition of this cascade in particular by a MEK inhibitor considerably inhibits the replication of this virus group, without having a toxic effect on the cells.

An improved therapy against DNA and RNA viruses the multiplication of which is dependent on the activity of the Raf/MEK/ERK cascade, is preferably directed therefore to this signaling pathway. It has been found that this signaling pathway is blocked by the application of a non-toxic pharmacological inhibitor. This non-toxic pharmacological inhibitor of the Raf/MEK/ERK signaling pathway is according to the invention a cascade inhibitor, in particular a MEK inhibitor.

### PRIOR ART

Virus infections are a considerable risk for the health of man and animal. In particular infections with the influenza A virus still belong to the big epidemics of mankind and are responsible year for year not only for a multitude of fatalities, but are also an immense cost factor for the whole economy, for instance by absence from work due to diseases [12].

Of equally important economic significance are infections with the Borna disease virus (BDV), in particular infecting horses and sheep, however also having been isolated from man already and being connected with neurological diseases [3, 13].

The problem of controlling RN viruses is the adaptability of the viruses caused by a high error rate of the viral polymerases, thus the production of suitable vaccines and also the development of antiviral substances being very difficult.

It has been shown that the application of antiviral substances immediately directed against functions of the virus, will very quickly lead to the selection of resistant variants, after a mutation. An example for this is the anti-influenza agent amantadine and the derivatives thereof being directed against a transmembrane protein and leading within a few passages already to the generation of resistant variants. The new anti-influenza therapeutic agents inhibiting the influenza-viral surface protein neuraminidase and being sold under the tradename RELANZA by Glaxo Wellcome in Germany, also have also produced variants already in patients [10]. Hopes being connected with this therapeutic agent could therefore not be fulfilled.

Due to the in most cases small genomes and thus limited coding capacity for functions being necessary for replication, all viruses are to a large extent dependent on the functions of their host cells. By influencing such cellular functions being necessary for the viral replication, it is possible to affect in a negative way the virus replication in the infected cell. There is no possibility for the virus to replace the missing cellular function by adaptation. An escape from the selection pressure by mutation is here not possible. This could already be shown for the example of the influenza A virus with relatively unspecific inhibiting substances against cellular kinases and methyl transferases [18].

It is the drawback in particular of these inhibiting substances that they have a relatively unspecific and broad effect, and that their cellular attacking points are only poorly defined. They are therefore not suitable for use as therapeutic agents. This is the problem: Until today, there are no inhibiting substances of cellular enzymes having a selective effect at this point without being toxic for the cell, as well as inhibiting the viral replication in particular of RNA viruses, such as Borna viruses or influenza A viruses.

With regard to the cellular processes induced after a virus infection, it is found that a multitude of DNA and RNA viruses activate in the infected host cell a defined signal transduction pathway, the so-called Raf/MEK/ERK kinase cascade [2, 4, 14, 17].

This kinase cascade belongs to the most important signaling pathways in the cell and plays an essential role in proliferation and differentiation processes.

Growth-factor induced signals are transferred by successive phosphorylation from the serine/theorine kinase Raf to the dual specific kinase MEK (MAP kinase kinase/ERK kinase) and finally to the kinase ERK (extracellular signal regulated kinase). Whilst as a kinase substrate of Raf, only MEK is known, and the ERK isoforms have been identified for MEK as the only substrate, ERK can phosphorylate quite a number of substrates. Hereto belong for instance the phosphorylation of transcription factors, which leads to a direct modification of the cellular gene expression [5, 15, 20].

The investigation of this signaling pathway in cellular decision processes has led to the identification of several pharmalogical inhibitors, which inhibit the signaling pathway, among other positions, on the level of MEK, i.e. at the ‘bottleneck’ of the cascade [1, 5, 7, 9].

The MEK inhibitor PD98059 (2-2′-amino-3′-methoxyphenyl)-oxanaphthalene-4-on [7] inhibits the activation of MEK by the kinase Raf.

The MEK inhibitor U0126 (1,4-diamino-2,3-dicyano-1,4-bis[2-aminophenylthio]butadiene) has been described as a substance partially inhibiting the activation of AP-1 dependent gene expression [9] and the proliferation of T cells [6].

In contrast to PD98059, the U0126 inhibits not only the MEK activation, but also the activity of the kinase itself [8].

Finally, the MEK inhibitor PD184352 has been described (2-(2-chloro-4-iodo-phenylamino)-N-cyclopropylmethoxy-3,4-difluoro-benzamide) [19], which with oral administration in the mouse model could efficiently inhibit the growth of colon carcinoma, without showing any significant signs of toxicity up to a cumulating dose of 6 g/kg body weight.

### OBJECT OF THE INVENTION

The invention is based on the object to provide substances for application in the prevention or therapy against intranuclear-replicating negative strand viruses, such substances not being immediately directed against functions of the virus, but selectively inhibiting a cellular enzyme, and inhibiting via this selective effect the viral replication of viruses.

Surprisingly, it has been found that this object can be achieved by a cascade inhibitor according to the invention or in particular by drugs containing a MEK inhibitor according to claim 1.

The cascade inhibitor according to the invention, in particular a MEK inhibitor, is a substance characterized by that it inhibits in a “cascade assay for inhibitors of the Raf/MEK/ERK kinase signaling pathway” the signaling cascade in vitro and in an “in vivo MEK and MAP kinase assay” the signaling cascade in vivo.

Cascade Assay for Inhibitors of the Raf/MEK/ERK Kinase Signaling Pathway.

For this cascade assay, the effect of inhibitors on the Raf/MEK/ERK signaling pathway is measured by kinase-arranged integration of radioactive 32P in the myelin basic protein (MBP) in presence of a 6xhistidine fusion protein of ERK (his-ERK) and a glutathione S-transferase fusion protein of MEK (GST-MEK).

The reaction mixture contains the recombinant proteins in a buffer of 20 mM HEPES, pH 7.4, 10 mM MgCl2, 1 mM MnCl2, 1 mM EGTA and 50 mM 32P-gamma-ATP in a total volume of 100 μl. The reaction takes 15 min at 30° C. and is stopped by addition of 20 μl Laemmli buffer. The radioactive-marked proteins were separated by SDS-PAGE and made visible by a phospho imager. Cascade inhibitors were tested in a concentration of 5-20 μl for their inhibiting ability in this assay. In order to differentiate whether a composition in this assay is a MEK or ERK inhibitor, the substances are tested in a second experimental approach with MBP and his-ERK under the above reaction conditions in presence of GST-MEK. A composition being effective in the first approach and having no effect in the second approach, is a MEK inhibitor. A composition being effective in the second approach and having no effect in the first approach, is an ERK inhibitor. A substance not being effective in any of the two approaches, having an effect however in the following in vivo MEK and MAP kinase assay, is a Raf inhibitor. All described inhibitors are according to the invention cascade inhibitors.

In Vivo MEK and MAP Kinase Assay.

Cells were sown in 10 cm cell culture dishes and grow to 80% confluence in cell culture medium with 10% fetal calf serum. The serum was removed for 8-12 h from the cells. Then the addition of the cascade inhibitors, in particular of the MEK inhibitors, is made, 30 min before the mitogenic stimulation of the cells, for instance with 100 ng/ml TPA or 100 ng/ml PDGF. After 10 min incubation with the mitogenic stimuli, the cells are washed with PBS and lysated in triton lysis buffer. (20 mM Tris pH 7.4, 50 nm Na-β-glycerol phosphate, 20 mM Na pyrophosphate, 137 mM NaCl, 10% (v/v) glycerin, 1% (v/v) triton X100, 2 mM EDTA, 1 mM Pefabloc, 1 mM Na-vanadate, 5 mM benzamidine, 5 μg/ml Aprotinin, 5 μg/ml Leupeptin). From these cell lysates, endogenous MEK is immuno-precipitated with a MEK-specific antiserum and incubated in an immune-complex kinase assay in presence of 32P-gamma-ATP, 0.1 mM ATP and recombinant kinase-inactive his-ERK K>M as the substrate protein at 30° C. for 15 min in a buffer of 10 mM MGCl2, 25 mM β-glycerol phosphate, 25 mM HEPES pH 7.5, 5 mM benzamidine, 0.5 ml DTT and 1 mM Na vanadate. Simultaneously, from the same lysate is immuno-precipitated endogenous ERK with a specific ERK antiserum and purified MBP under the same conditions as MEK. The proteins are dissociated on a SDS-PAGE gel and visualized by means of a phospho imager. A cascade inhibitor, in particular a MEK inhibitor acts in this assay in an inhibiting way on the MEK activation, as measured by the phosphorylation of his-ERK K>M, as well as on the ERK activation, as measured by the phosphorylation of MBP.

The application according to the invention of the cascade inhibitors, in particular of the MEK inhibitors, relates in particular to the following substances:
a) 2-(2-Amino-3-methoxyphenyl)-4-oxo-4H-(1)benzopyran (as also described in WO 98/37881)
b) 1,4-Diamino-2,3-dicyano-1,4-bis[2-aminophenylthio]butadiene (short designation: U0126)
c) 2-(2-chloro-4-iodo-phenylamino)-N-cyclopropylmethoxy-3,4-difluoro-benzamide) (short-form designation: PD18453)
d) 2-(2′-amino-3′-methoxyphenyl-oxanaphthalene-4-on (short-form designation: PD98059)
e) substances characterized by that they act as cascade inhibitors according to the invention and originate in particular from the chemical substance classes of the butadiene derivatives or flavin derivatives or benzamide derivatives,
f) all derivatives of the aforementioned substances acting as cascade inhibitors, in particular MEK inhibitors,
g) further substances acting as cascade inhibitors, in particular MEK inhibitors (pre-stage substances, salts or “prodrugs” in the meaning of [11, 16] of the afore-mentioned compositions or their derivatives, the effectiveness of which in the cascade assay for inhibitors of the Raf/MEK/ERK signaling pathway or in the “in vivo MEK and MAP kinase assay” is proven).

The invention relates to the application of these substances as drugs for patients being infected with a DNA or RNA virus, in particular an intranuclear-replicating negative strand RNA virus, for instance an influenza A virus or a Borna disease virus.

In another type of the application according to the invention, it is suggested to use drugs comprising these substances for the prevention of an infection with a DNA or RNA virus, in particular an intranuclear-replicating negative strand RNA virus, for instance an influenza A virus or a Borna disease virus.

The term patient relates equally to human beings and vertebrates. Thus the drugs can be used in human and veterinary applications. The therapeutically effective substances of the present invention are administered to the patients as part of a pharmaceutically acceptable composition either in an oral, rectal, parenteral-intravenous, intramuscular or subcutaneous, intracisternal, intravaginal, intraperitoneal, intravascular, local (powder, ointment or drops) or spray form.

Pharmaceutically acceptable compositions may contain the modifications as salts, esters, amides and “prodrugs”, as far as they will not, after a reliable medical evaluation, cause excessive toxicity, irritations or allergic reactions of the patient.

The term “prodrug” relates to compositions being transformed for a better reception, as for instance by hydrolysis in blood. A detailed discussion is given in [11] and [16].

Dosing types for the local administration of the composition of the invention include ointments, powder, sprays or inhalation means. The active component is mixed under sterile conditions with a physiologically acceptable carrier and possible preservatives, buffers or driving means, depending on the necessity.

### EXAMPLES

The example 1 shows for the MEK inhibitor U0126 that with increasing concentration of the inhibitor U0126 in the cell culture medium, the number of the newly generated infectious influenza A virus particles is significantly reduced.

For the multiplication of influenza A viruses, permissive eukaryotic cell cultures (Madine-Darby canine kidney (MDCK) cells), were washed in parallel approaches having equal cell counts with a physiological salt solution and infected with an equal amount of the infectious influenza A virus stem WSN-HK (reassortant having seven gene segments of influenza stem A/WSN/33 and the NA gene of influenza stem A/HK/8/68), in a ratio of 0.0025 infectious virus particles per cell for one hour at room temperature.

30 min before the infection, the MDCK cells are incubated in a suitable cell culture medium being reacted in different concentrations with the MEK inhibitor U0126 (0 μM, 30 μM, 40 μM, 50 μM dissolved in DSMO) at 37° C. and 5% CO₂. As a solvent reference, MDCK cells were incubated with cell culture medium reacted with the corresponding various amounts of DMSO. During the infection, the MEK inhibitor U0126 or DMSO as a solvent is added to the inoculum in the corresponding concentrations.

Subsequently, the inoculum is removed, and the infected cells are incubated in a suitable cell culture medium being reacted in different concentrations with the MEK inhibitor U0126 (0 μM, 30 μM, 40 μM, 50 μM dissolved in DSMO) for 48 h at 37° C. and 5% CO₂. As a solvent reference, MDCK cells were incubated with cell culture medium reacted with the corresponding various amounts of DMSO. 24 hours after the infection, 200 μl of the medium supernatant were removed, and the same volume of inhibitor or DMSO-containing cell culture medium were re-added to the medium supernatant. After 48 h, another sample was taken. The cell culture supernatants of the respective samples for the 24 and the 48 h value are examined to conventional virological methods for the amount of hemagglutinating units (HA titer) representing the total production of virus particles, and for the amount of newly generated infectious virus particles (plaque assay on MDCK cells).

As a result, it can be found in such an experimental approach that with increasing concentration of the MEK inhibitor U0126 the number of newly generated infectious virus particles is significantly reduced (approx. 80% for 50 μM U0126) in the cell culture medium, compared to the reference approach without MEK inhibitor U0126 or the solvent references, respectively. The macroscopic examination of MDCK cells treated with corresponding concentrations of DMSO or MEK inhibitor U0126 dissolved in DMSO, as well as a cytotoxicity examination by means of propidium iodide staining show that neither solvent nor inhibitor have a significant cytotoxic effect on the cells.

The example 2 shows that with increasing concentration of the MEK inhibitor U0126 in cell culture medium also the number of newly generated infectious Borna disease viruses particles is significantly reduced.

Cells pre-treated with inhibitor are infected with BDV, and the spreading of the infection is observed in an indirect immunofluorescence against the viral nucleoprotein. After a one-time administration of 25 μM MEK inhibitor (U0126), no virus foci are visible after a cultivation time of 7 days, but only individual infected cells. After an administration of 12.5 μM kinase inhibitor (U0126), the effect is not clear anymore, and after an administration of 6 μM kinase inhibitor (U0126), no difference of the virus foci can be found compared to untreated infectious reference cells. The inhibitor acts therefore in a dosage-dependent manner on the level of the virus replication.

The inhibitory effect of the MEK inhibitor (U0126) in the described applications shows that the cascade inhibitors, in particular MEK inhibitors, can be used as antiviral agents against influenza and Borna viruses in particular, however also against RNA and DNA viruses, for which a dependence of the viral multiplication on the activity of the Raf/MEK/ERK cascade exists. The signaling path is herein according to the invention the aim of the antiviral therapy and is preferred by application of a non-toxic pharmacological cascade inhibitor, in particular a MEK inhibitor.

Literature.

[1] Alessi, D. R., Cuenda, A., Cohen, P., Dudley, D. T., and Saltiel, A. R. (1995). PD 098059 is a specific inhibitor of the activation of mitogen-activated protein kinase kinase in vitro and in vivo J. Biol. Chem. 270, 27489-27494.
[2] Benn, J., Su, F., Doria, M., and Schneider, R. J. (1996). Hepatitis B virus Hex protein induces transcription factor AP-1 by activation of extracellular signal-regulated and c-Jun N-terminal mitogen-activated protein kinases. J. Virol, 70, 4978-4985.
[3] Bode, L., Zimmermann, W., Ferszt, R., Steinbach, F., and Ludwig, H. (1995). Borna disease virus genome transcribed and expressed in psychiatric patients (see comments). Nat Med I. 232-6.
[4] Bruder, J. T., and Kovesdi, I. (1997). Adenovirus infection stimulates the Raf/MAPK signaling pathway and induces interleukin-S expression. J. Virol. 71, 398-404.
[5] Cohen, P. (1997). The search for physiological substrates of MAP and SAP kinases in mammalian cells. Trends in Cell Biol. 7, 353-361.
[6] DeSilva, D. R., Jones, E. A., Favata, M. F., Jaffee, B. D., Magolda, R. L., Trzaskos, J. M., and Scherle, P. A. (1998). Inhibition of mitogen-activated protein kinase kinase blocks T cell proliferation but does not induce or prevent anergy. J. Immunol. 160, 4175-4181.
[7] Dudley, D. T., Pang, L., Decker, S. J., Bridges, A. J., and Saltiel, A. R. (1995). A synthetic inhibitor of the mitogen-activated protein kinase cascade. Proc. Natl, Acad. Sci. USA 92, 7686-7689.
[8] Duncia, J. V., Santella, J. B. r., Higley, C. A., Pitts, W. J., Wityak, J., Frietze, W. E., Rankin, F. W., Sun, J. H., Earl, R. A., Tabaka, A. C., Teleha, C. A., Blom, K. F., Favata, M. F., Manos, E. J., Daulerio, A. J., Stradley, D. A., Horiuchi, K., Copeland, R. A., Scherle, P. A., Trzaskos, J. M., Magolda, R. L., Trainor, G. L., Wexler, R. R., Hobbs, F. W., and Olson, R. E. (1998). MEK inhibitors; the chemistry and biological activity of U0126, its analogs, and cyclization products. Bioorg Med Chem Lett 8, 2839-44.
[9] Favata, M. F., Horiuchi, K. Y., Manos, E. J., Daulerio, A. J., Stradley, D. A., Feeser, W. S., Van Dyk, D. E., Pitts, W. J., Earl, R. A., Hobbs, F., Copeland, R. A., Magolda, R. L., Scherle, P. A., and Trzaskos, J. M. (1998). Identification of a novel inhibitor of mitogen-activated protein kinase kinase. J. Biol. Chem. 273, 18623-18632.
[10] Gubareva, L. V., Matrosovich, M. N., Brenner, M. K., Bethell, R. C., and Webster, R. G. (1998). Evidence for zanamivir resistance in an immunocompromised child infected with influenza B virus. J Infect Dis 178, 1257-62.
[11] Higuchi, T., and Stella, V. (1987). Prodrugs as novel delivery systems, In A.C.S. Symposium Series.
[12] Lamb, R. A., and Krug, R. M. (1996). Orthomyxoviridae: The viruses and their replication. In Fields Virology, B. N. e. a. Fields, ed. (Philadelphia: Lippincott-Raven Publishers), pp. 1353-1395.
[13] Planz, O., Rentzsch, C., Batra, A., Winkler, T., Buttner, M., Rziha, H. J., and Stitz, L. (1999). Pathogenesis of borna disease virus: granulocyte fractions of psychiatric patients harbor infectious virus in the absence of antiviral antibodies. J Virol 73, 6251-6.
[14] Popik, W., and Pitha, P. M. (1998). Early activation of mitogen-activated protein kinase kinase, extracellular signal-regulated kinase, p38 mitogen-activated protein kinase, and c-Jun N-terminal kinase in response to binding of simian immunodeficiency virus to Jurkat T cells expressing CCR5 receptor. Virology 252, 210-217.
[15] Robinson, M. J., and Cobb, M. H. (1997). Mitogen-activated protein kinase pathways. Curr. Opin. Cell Biol. 9, 180-186.
[16] Roche, E. B. E. (1987). Bioreversible Carriers in Drug Design, E. B. Roche, ed.: American Pharmaceutical Association and Pergamon Press.
[17] Rodems, S. M., and Spector, D. H. (1998). Extracellular signal-regulated kinase activity is sustained early during human cytomegalovirus infection. J. Virol. 72, 9173-9180,
[18] Scholtissek, C., and Muller, K. (1991). Failure to obtain drug-resistant variants of influenza virus after treatment with inhibiting doses of 3-deazaadenosine and H7. Arch Virol. 119, 111-118.
[19] Sebolt-Leopold, J. S., Dudley, D. T., Herrera, R., Van Becelaere, K., Wiland, A, Gowan, R. C., Tecle, H., Barrett, S. D., Bridges, A., Przybranowski, S., Leopold, W. R., and Saltiel, A. R. (1999). Blockade of the MAP kinase pathway suppresses growth of colon tumors in vivo. Nature Med. 5, 810-816.
[20] Treisman, R. (1996). Regulation of transcription by MAK kinase cascades. Curr. Opin. Cell Biol. 8, 205-215.

1. A cascade inhibitor, that is an MEK inhibitor, selected from the group consisting of butadiene derivatives, flavin derivatives, and benzamide derivatives.

2. A method of using a cascade inhibitor, that is an MEK inhibitor, to prevent or treat infections caused by DNA or RNA viruses comprising administering in a mammal a compound selected from the group consisting of butadiene derivatives, flavin derivatives, and benzamide derivatives.

3. A method of claim 2 further comprising the compounds selected from the group consisting of:
a) 2-(2-Amino-3-methoxyphenyl)-4-oxo-4H-(1)benzopyran, 1,4 Diamino-2,3-dicyano-1,
b) 1,4-Diamino-2,3-dicyano-1,
c) 4-bis[2-aminophenylthio]butadiene,
d) 2-(2-chloro-4-iodo-phenylamino)-N-cycloopropyl-methoxy-3,4-difluorobenzamide,
e) 2-(2′-amino-3′-methoxyphenyl)-oxanaphthalene-4-on;
or pre-stage substances, a pharmaceutically acceptable salt thereof, prodrugs, derivatives or mixtures thereof.

4. The method of claim 2, wherein the compound is used to treat the infections caused by negative strand RNA viruses in mammals.

5. The method of claim 2, wherein the compound is used to treat the infections caused by intranuclear-replicating negative strand RNA viruses in mammals.

6. The method of claim 2, wherein the compound is used to treat the infections caused by Influenza A and Borna Viruses in mammals.

7. The method of claim 5, wherein the compound is an MEK inhibitor.

8. The method of claim 7, wherein the MEK inhibitor is 2-(2-Amino-3-methoxyphenly)-4-oxo-4H-(1)benzopyrane, PD18453, PD98059 or U0126.

9. A method for preventing a mammal from being infected with an intranuclear-replicating negative strand RNA virus, wherein a compound comprising an MEK inhibitor is administered to a patient.

10. The method to claim 9 wherein, the MEK inhibitor is 2-(2-Amino-3-methoxyphenyl)-4-oxo-4H-(1)benzopyrane, PD18453, PD98059 or U0126.

11. The method of claim 7 wherein the virus is an Influenza A Virus or a Borna Disease Virus.

12. The method of claim 8 wherein the virus is an Influenza A Virus or a Borna Disease Virus.

13. The method of claim 9 wherein the virus is an Influenza A Virus or a Borna Disease Virus.

14. The method of claim 10 wherein the virus is an Influenza A Virus or a Borna Disease Virus.
